(19) United States
(12) Patent Application Publication
(10) Pub. No.: US 2019/0262314 A1
(43) Pub. Date: Aug. 29, 2019

(54) READY-TO-USE DEXMEDETOMIDINE COMPOSITIONS

(71) Applicant: SLAYBACK PHARMA LLC, Princeton, NJ (US)

(72) Inventors: Ashish Anil Dubewar, Hyderabad (IN); Pradeep Kumar Kare, Hyderabad (IN); Kumar Swamy Ummiti, Hyderabad (IN); Ajay Kumar Singh, Princeton, NJ (US)

(21) Appl. No.: 16/276,885

(22) Filed: Feb. 15, 2019

(30) Foreign Application Priority Data
Feb. 26, 2018 (IN) 201841007136
Dec. 5, 2018 (IN) 201841046015

(51) Int. Cl. A61K 31/4174; A61K 47/20; A61K 9/00; A61K 9/08; A61K 47/02; A61J 1/14

(52) U.S. Cl. CPC A61K 31/4174; A61K 47/20; A61J 1/1468; A61K 9/08; A61K 47/02; A61K 9/0019

(57) ABSTRACT

The present invention provides stable, ready-to-use pharmaceutical compositions, comprising dexmedetomidine or a pharmaceutically acceptable salt thereof, one or more pharmaceutically acceptable excipients, and a pharmaceutically acceptable liquid vehicle, where the composition is provided in a sealed container that comprises a cyclic olefin polymer. The present invention also provides ready-to-use pharmaceutical compositions comprising dexmedetomidine or a pharmaceutically acceptable salt thereof, a pharmaceutically acceptable liquid vehicle and an antioxidant and/or stabilizer, such as L-methionine. These solutions were found to be stable over the shelf life of the product. Other aspects of the invention relate to methods for making such compositions, as well as methods of using such compositions for perioperative care of a patient or for sedation.

### CROSS-REFERENCE TO RELATED APPLICATIONS

This application claims foreign priority to Indian Application Nos. IN 201841007136, filed on Feb. 26, 2018, and IN 201841046015, filed on Dec. 5, 2018, which are incorporated herein by reference in their entirety.

### FIELD OF THE INVENTION

The present invention relates to stable, ready-to-use injectable compositions comprising dexmedetomidine, or a pharmaceutically acceptable salt thereof, where the finished dosage form is provided in a sealed polymeric container (e.g., vials, pre-filled syringes, infusion bags, bottles, ampoules, etc.), and where the polymeric container comprises a cyclic olefin polymer. The present application also provides antioxidant-containing compositions of dexmedetomidine provided in a polymeric container, which comprises a cyclic olefin polymer. These pharmaceutical compositions are suitable for injectable administration (e.g., subcutaneous, intravenous or intramuscular), and are stable over the shelf life of the product.

Other aspects of the invention relate to methods for making such compositions, as well as methods of treatment using such compositions. The pharmaceutical compositions of the present application are useful for perioperative care of a patient or for sedation.

### BACKGROUND OF THE INVENTION

The present invention relates to stable, ready-to-use, injectable compositions, comprising dexmedetomidine, or a pharmaceutically acceptable salt thereof. Dexmedetomidine hydrochloride is the S-enantiomer of medetomidine and is chemically described as (+)-4-(S)-[1-(2,3-dimethylphenyl) ethyl]-1H-imidazole monohydrochloride. Medetomidine is a selective and potent α2-adrenoceptor agonist, which is used as an antihypertensive agent and as a sedative-analgesic agent.

Dexmedetomidine is commercially available under tradename PRECEDEX™ as a 200 mcg/2 mL solution for injection in a glass vial, to be used after dilution. It is also available as a ready-to-use solution in 0.9% sodium chloride at a concentration of 4 mcg/mL, which is provided in 20 mL, 50 mL and 100 mL glass bottles.

Dexmedetomidine hydrochloride is represented by the following structural formula (I):

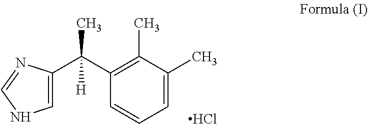

U.S. Pat. Nos. 5,344,840 and 5,091,402 disclose the use of dexmedetomidine in perioperative and epidural applications, respectively. When used in perioperative care, dexmedetomidine can reduce the amount of anaesthetic necessary to anesthetize a patient. U.S. Pat. No. 6,716,867 discloses methods of sedating a patient in an intensive care unit by administering dexmedetomidine, or a pharmaceutically acceptable salt thereof, to a patient in need thereof.

Developing ready-to-use injectable compositions comprising dexmedetomidine has been a challenge due to incompatibilities and/or interactions with certain materials, resulting in a limited selection of appropriate materials for the containers. U.S. Pat. No. 8,242,158 discloses premixed dexmedetomidine solutions packaged in glass vials, because of the tendency of dexmedetomidine to be either adsorbed or absorbed by plastic materials. U.S. Pat. No. 9,717,796 discloses premixed, ready-to-use dexmedetomidine solution packaged in a flexible plastic container that is substantially free of DEHP.

Currently, commercially-available dexmedetomidine drug products are provided in glass ampoules or vials, which have several drawbacks including the risk of breakage, glass particulate issues, difficulty in shipping/transporting, and their heavy weight, as well as potential delamination, reactivity or leaching. Consequently, there is a need for alternative formulations and finished drug products, with improved stability, which are conveniently provided in a commercially available container closure.

Thus, there exists a need for the development of novel or improved compositions comprising dexmedetomidine that are stable and/or can be provided as ready-to-use pharmaceutical compositions. Providing a ready-to-use product will advantageously improve patient compliance, reduce dosage errors, and reduce risk of contamination.

### SUMMARY OF THE INVENTION

In one embodiment, the present application relates to a ready-to-use, injectable pharmaceutical composition comprising (i) dexmedetomidine or a pharmaceutically acceptable salt thereof, (ii) at least one pharmaceutically acceptable excipient and (iii) at least one pharmaceutically acceptable liquid vehicle, wherein the composition is provided in a sealed container, and wherein the sealed container comprises a cyclic olefin polymer.

In another embodiment, the present application provides ready-to-use compositions comprising dexmedetomidine or a pharmaceutically acceptable salt thereof and an antioxidant. In another aspect, the present application provides ready-to-use compositions comprising dexmedetomidine or a pharmaceutically acceptable salt thereof and an antioxidant, provided in a polymeric container that comprises a cyclic olefin polymer. In a further embodiment, the present application provides storage stable, ready-to-use compositions comprising dexmedetomidine or a pharmaceutically acceptable salt thereof and an antioxidant, provided in a sealed container that comprises a cyclic olefin polymer.

The sealed container comprises a cyclic olefin polymer in at least one contact layer, wherein the contact layer is in direct contact with the pharmaceutical composition. For example, the sealed container may be selected from the group consisting of vials, pre-filled syringes, bags, bottles and ampoules, and may be made from a cyclic olefin polymer, or comprise a cyclic olefin polymer in at least one contact layer.

In particular, the cyclic olefin may be a polymer selected from the group consisting of cyclic olefin copolymers, cyclic olefin polymers, and mixtures thereof. In certain non-limiting embodiments, the polymeric container is selected from CZ® (Crystal Zenith) resin containers and syringes (available form West Pharmaceutical Services, Inc.). In certain non-limiting embodiments, the polymeric container may be selected from laminated bags having an inner layer comprising a cyclic olefin copolymer (e.g., POLYELITE® PHC three-layered bags, which are commercially available from Hosokawa Micron Corporation, Japan).

In particular, the pharmaceutical composition according to the present application comprises dexmedetomidine hydrochloride. Preferably, the dexmedetomidine concentration is about 4 μg/mL.

The pharmaceutical composition may be formulated at a pH of between about 4.5 and about 7.0.

The pharmaceutical compositions may comprise one or more pharmaceutically acceptable excipients. For example, the compositions may further comprise a tonicity adjusting agent, a preservative, a pH adjusting or neutralizing agent, an antioxidant, and/or a stabilizer.

In particular, the pharmaceutical composition described in the present application may further comprise an antioxidant or a stabilizer. In certain embodiments, the pharmaceutical composition according to application may comprise a stabilizing amount of methionine, glycerol, propylene glycol, phenol, EDTA, BHT, or mixtures thereof. In certain embodiments, the compositions comprise L-methionine. In certain preferred embodiments, an antioxidant is provided in a stabilizing amount. For example, the antioxidant may be provided in an amount of about 0.05 mg/mL to about 5 mg/mL.

The present application provides a storage stable, ready-to-use composition comprising dexmedetomidine or a pharmaceutically acceptable salt thereof, a tonicity adjusting agent and an antioxidant in a polymeric container comprising a cyclic olefin polymer. In a further embodiment, the present application provides storage stable, ready-to-use compositions comprising dexmedetomidine or a pharmaceutically acceptable salt thereof, a tonicity adjusting agent and methionine in a polymeric container comprising a cyclic olefin polymer.

In yet another embodiment, the present application provides a storage stable, ready-to-use composition comprising dexmedetomidine or a pharmaceutically acceptable salt thereof, a tonicity adjusting agent and methionine in a polymeric container, where the polymer that is in contact with the composition comprises a cyclic olefin copolymer. In a related embodiment, the present application provides a storage stable, ready-to-use composition comprising dexmedetomidine or a pharmaceutically acceptable salt thereof, sealed in a polymeric container, where the polymer that is in contact with the composition comprises cyclic olefin copolymer.

In various embodiments, the application relates to ready-to-use pharmaceutical composition comprising (i) dexmedetomidine or a pharmaceutically acceptable salt thereof, (ii) an antioxidant, and (iii) a pharmaceutically acceptable liquid vehicle. In certain embodiments, the pharmaceutical compositions comprise from about 0.005 μg/mL to about 10 μg/mL of dexmedetomidine or a pharmaceutically acceptable salt thereof; and from about 0.05 mg/mL to about 2 mg/mL of an antioxidant. Certain pharmaceutical compositions of the present application, comprise about 4 μg/mL of dexmedetomidine; about 9.0 mg/mL of sodium chloride; about 1.0 mg/mL of L-methionine; and water for injection.

In embodiments of the invention, the pharmaceutical compositions are stable for 6 months when stored at ambient conditions. In certain embodiments, the pharmaceutical compositions are stable for at least 6 months at 25° C. and 60% relative humidity. In certain embodiments, the pharmaceutical compositions are stable for at least 3 months at 40° C. and 75% relative humidity.

Certain methods for making a pharmaceutical composition according to application comprise (i) combining one or more antioxidants or stabilizers with water for injection to form a first solution; (ii) adding dexmedetomidine or a pharmaceutically acceptable salt thereof to the first solution to provide a second solution; (iii) optionally, adding a tonicity adjusting agent to the second solution to provide a third solution; (iv) optionally, adjusting the pH of the third solution by adding hydrochloric acid or sodium hydroxide to provide a fourth solution; (v) filling one or more containers with the fourth solution; and (vi) sealing the containers; and optionally, sterilizing the containers. A pharmaceutically inert gas (e.g., which may be selected from nitrogen or carbon dioxide) may be bubbled into the solution to drive out oxygen. In certain aspects, rubber stoppers may be used for sealing the vials. Certain embodiments additionally relate to sterilizing the finished products, e.g., filtration through a bacterial-retaining filter, aseptic filling, terminal sterilization, incorporation of sterilizing agents, irradiation, and/or heating.

In certain embodiments, ready-to-use compositions of the present application are used in methods of treatment for the perioperative care of a patient or for sedation. Certain embodiments of the invention relate to methods of treatment, comprising administering the pharmaceutical compositions to a patient in need thereof, for perioperative care or for sedation.

### BRIEF DESCRIPTION OF THE FIGURES

FIG. 1 shows the impurity profile by HPLC for the composition of Example 5, when stored for 3 months at 40° C. and 75% relative humidity (RH).

FIG. 2 shows the impurity profile by HPLC for the composition of Example 6, when stored for 3 months at 40° C. and 75% relative humidity (RH).

FIG. 3 shows the impurity profile by HPLC for the composition of the Comparative Example at Table 10, when stored for 3 months at 40° C. and 75% relative humidity (RH).

FIG. 4 shows the impurity profile by HPLC for the composition of the Comparative Example at Table 11 when stored for 3 months at 40° C. and 75% relative humidity (RH).

FIG. 5 shows the impurity profile by HPLC for the composition of the Comparative Example at Table 12, when stored for 3 months at 40° C. and 75% relative humidity (RH).

### DETAILED DESCRIPTION OF THE INVENTION

In one embodiment, the present application relates to a ready-to-use pharmaceutical composition comprising (i) dexmedetomidine or a pharmaceutically acceptable salt thereof, (ii) at least one pharmaceutically acceptable excipient and (iii) at least one pharmaceutically acceptable liquid vehicle, wherein the composition is provided in a sealed container, and where the sealed container comprises a cyclic olefin polymer.

In another embodiment, the present application provides a ready-to-use composition comprising dexmedetomidine, or a pharmaceutically acceptable salt thereof, and an antioxidant. In another embodiment, the present application provides ready-to-use composition comprising dexmedetomidine, or a pharmaceutically acceptable salt thereof, and an antioxidant in a polymeric container, which contains a cyclic olefin polymer.

In certain embodiments, sealed polymeric containers (e.g., vials, pre-filled syringes, bags, bottles and ampoules) are used, wherein the sealed polymeric container comprises a cyclic olefin polymer. In certain embodiments, the cyclic olefin polymer is at least present in a contact layer, e.g., in direct contact with the pharmaceutical composition.

As used herein, the term “dexmedetomidine” comprises all pharmaceutically acceptable salts, solvates, prodrugs, complexes, or hydrates thereof, as well as any polymorphic or amorphous forms or combinations thereof. The term “dexmedetomidine” also refers to the corresponding free base form. Pharmaceutically acceptable salts of dexmedetomidine include inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, and salicylic acid. Preferred forms of dexmedetomidine include dexmedetomidine hydrochloride.

Within the context of the present invention, the term “ready-to-use” or “RTU” as used herein refers to a pharmaceutical composition that is stable and is not reconstituted from a lyophilizate. In certain embodiments, for example, the ready-to-use pharmaceutical composition is an injectable composition that is premixed and suitable for administration to a patient without dilution. The term “RTU” encompasses within its scope, parenteral compositions that are stable and must be diluted from a concentrated, liquid solution just prior to use. The term “RTU” also encompasses formulations that are premixed. As used herein the terms “premix” or “premixed” refer to a pharmaceutical composition, which does not require reconstitution or dilution prior to administration to a patient, e.g., suitable for parenteral administration. In certain embodiments, the compositions of the present invention are “ready to use” upon removing the compositions from a sealed container or vessel.

The pharmaceutical compositions of the present invention provide ready-to-use injectable compositions, whereby there is no requirement for reconstituting the composition prior to its administration, thus eliminating the tedious task of reconstitution, especially in an aseptic area. As such, the compositions of the present invention provide a convenient dosage form, improve patient compliance, reduce dosage errors, and reduce risk of contamination. Advantageously, in certain embodiments, the need for lyophilization and/or formation of a lyophilized solid during manufacturing can also be avoided.

The pharmaceutical compositions described herein may be in any form suitable for injection. To prepare such compositions, active drug(s) are dissolved or suspended in a parenterally acceptable liquid vehicle. In certain non-limiting embodiments, the ready-to-use dexmedetomidine composition is formulated as a liquid and may be provided in the form of a solution, suspension, or emulsion. The pharmaceutically acceptable liquid vehicle or solvent may comprise water, saline, alcohols such as ethanol, polyol (for example, glycerol, propylene glycol, and polyethylene glycol, and the like), dimethylacetamide N-methylpyrolidone, dimethyl sulfoxide, Ringer's solution, isotonic sodium chloride solution, or suitable mixtures thereof.

The compositions of the invention can be administered in any conventional manner. It will be readily appreciated by those skilled in the art how to administer compositions of the present invention to a human or an animal. The formulation is preferably suitable for parenteral administration, including, but not limited to, intravenous, subcutaneous, intramuscular and intraperitoneal administration.

According to present invention, the ready-to-use composition of the present invention is in a form selected from solution, suspension, or emulsion suitable for parenteral administration comprising dexmedetomidine or a pharmaceutically acceptable salt thereof with one or more parenterally acceptable excipients.

In certain non-limiting embodiments, dexmedetomidine is formulated as a composition, wherein the dexmedetomidine is the only therapeutically active ingredient present in the composition. In another non-limiting embodiment, dexmedetomidine is formulated as a composition, wherein the dexmedetomidine is formulated in combination with at least one or more other therapeutically active ingredient.

In certain non-limiting embodiments, the ready-to-use dexmedetomidine composition comprises dexmedetomidine, or a pharmaceutically acceptable salt thereof, at a concentration of between about 0.005 μg/mL and about 10 μg/mL, or between about 0.005 μg/mL and about 7 μg/mL, or between about 0.005 μg/mL and about 5 μg/mL, or between about 0.005 μg/mL and about 4 μg/mL

In certain non-limiting embodiments, the ready-to-use dexmedetomidine composition comprises dexmedetomidine at a concentration of about 4 μg/mL.

As used herein, the term “storage” refers to the holding of a composition under controlled or uncontrolled conditions for a period ranging from a few minutes to several months or longer. Storage conditions that can be controlled include, for example, temperature, humidity, and the level of light. In many cases, storage of a pharmaceutical formulation is under industry acceptable standards and/or standards that are mandated by regulatory agencies, such as US FDA.

The term “stable” refers to both the physical and chemical stability of a composition in any form, such as a solution. A composition is said to be stable if it exhibits minimal change over time relative to when it is manufactured. Stability is measured at various time points through a planned product expiration date with evaluation criteria including such items as therapeutic activity, appearance, levels of particulate matter, pH, content of active ingredient(s), and levels of degradation products, impurities, or related substances. For purposes of the present invention, “stabilizing amount” shall be understood to include those amounts which increase or enhance the stability of the compositions described herein, as compared to a composition without the stabilizing agent.

The term “pharmaceutically acceptable excipient” as used herein means a diluent, carrier, or composition auxiliary, which is non-toxic and inert, which does not have undesirable effects on a subject to whom it is administered and is suitable for delivering a therapeutically active agent to the target site without affecting the therapeutic activity of the said active agent. In certain embodiments, the pharmaceutical compositions may optionally contain pharmaceutically acceptable excipients, including antioxidants, buffers, tonicity adjusting agents, preservatives, stabilizing agents, or pH adjusting agents.

In certain embodiments, the pharmaceutical compositions may optionally contain an antioxidant or stabilizing agent. The term “antioxidant” as used herein refers to the compounds or materials that have the tendency to prevent or inhibit oxidation, which thereby prevents degradation of dexmedetomidine. The term “stabilizing agent” means a compound that is used to stabilize a therapeutic agent against physical, chemical, or biochemical process that would otherwise reduce the therapeutic activity of the agent. Suitable stabilizers include, by way of example and without limitation, albumin, sialic acid, creatinine, niacinamide, sodium acetyltryptophonate, zinc oxide, sucrose, glucose, lactose, sorbitol, mannitol, glycerol, polyethylene glycols, sodium caprylate and sodium saccharin, as well as others known to those of ordinary skill in the art.

In certain preferred embodiments, the antioxidant is provided in a stabilizing amount. In certain non-limiting embodiments, the antioxidant or stabilizing agent may include methionine, glycerol, monothioglycerol, propylene glycol, phenol, EDTA or BHT. The term “methionine” as used herein encompasses both L-methionine and D-methionine. Examples of antioxidant and stabilizing agents may also include by way of example and without limitation, acetone, sodium bisulfate, ascorbic acid, ascorbyl palmitate, citric acid, glycine, L-cysteine hydrochloride, D-methionine, L-methionine, butylated hydroxy anisole, butylated hydroxytoluene, hydro phosphorous acid, monothioglycerol, propyl gallate, sodium ascorbate, sodium citrate anhydrous, sodium citrate dihydrate, sodium sulfide, sodium sulfite, sodium bisulfite, sodium formaldehyde sulfoxylate, thioglycolic acid, sodium metabisulfite and others known to those of ordinary skill in the art. The amount of antioxidant or stabilizing agent may range from about 0.01 mg/mL to about 50 mg/mL of the composition, preferably from about 0.05 mg/mL to about 5 mg/mL, and most preferably from about 0.05 mg/mL to about 2 mg/mL. In certain embodiments, the compositions comprise from about 1.0 mg/mL to about 1.25 mg/mL of methionine.

The pharmaceutical compositions may optionally contain a buffering agent, which is used to resist change in pH upon dilution or addition of acid or alkali. Such compounds include, by way of example and without limitation, acetic acid, sodium acetate, adipic acid, benzoic acid, sodium benzoate, maleic acid, monobasic sodium phosphate, dibasic sodium phosphate, disodium hydrogen phosphate dodecahydrate, lactic acid, tartaric acid, potassium metaphosphate, potassium phosphate, monobasic sodium acetate, sodium bicarbonate, sodium tartrate and others known to those of ordinary skill in the art.

The pharmaceutical compositions may contain a “tonicity modifier” that can be used to adjust the tonicity of the liquid formulation. Suitable tonicity modifiers include glycerine, lactose, mannitol, dextrose, sodium chloride, sodium sulphate, sorbitol, trehalose and others known to those of ordinary skill in the art. In one embodiment, the tonicity of the liquid formulation approximates that of the tonicity of blood or plasma. The amount of tonicity modifier may range from about 1 mg/mL to about 20 mg/mL of the composition, preferable from about 5-10 mg/mL. In certain embodiments, the composition may contain sodium chloride at a concentration of about 5 mg/mL to about 15 mg/mL, preferably sodium chloride at a concentration of about 5 mg/mL to about 10 mg/mL, more preferably, sodium chloride at a concentration of about 9 mg/mL. In certain embodiments, the composition will have an osmolality between about 200 to about 400 mOsm/kg, preferably between about 270 to about 340 mOsm/kg.

The present invention provides for a composition that may optionally comprise one or more preservatives. The term “preservative” refers to a substance present in a composition which can, at least in part, prevent and/or reduce decomposition of the composition. In some embodiments, the preservative may prevent and/or reduce decomposition of the composition by microbial growth in the composition. Preferably, the preservative is pharmaceutically acceptable.

In some embodiments, the preservative may be present in the composition at a concentration that allows for a multi-dose formulation of the composition. In some embodiments, the preservative may be present in the composition at a concentration that prevents and/or reduces decomposition of unused portions of the composition in a multi-dose formulation. In some embodiments, the preservative may allow for up to about 14 days of use, preferably up to about 30 days of use, more preferably up to about 60 days of use, and most preferably up to about 90 days of use of a multi-dose formulation of the composition.

In some embodiments, the preservative may be present in the composition at a concentration of in the range of about 1 to 10 mg/mL, preferably in the range of about 3 and 7 mg/mL, more preferably in the range of about 4 and 5 mg/mL, more preferably at about 4.5 mg/mL.

Preferably, preservatives comprise one or more of benzalkonium chloride, benzethonium chloride, benzoic acid, benzyl alcohol, benzyl paraben, bronopol, butyl paraben, cetrimide, cetylpyridinium chloride, chlorbutanol, chlorhexidine, chlorocresol, chloroxylenol, cresol, ethyl alcohol, ethyl paraben, ethylparaben, glycerin, hexetidine, imidurea, isobutyl paraben, meta-cresol, methyl paraben, methylparaben, phenol, phenoxyethanol, phenylethyl alcohol, phenylmercuric nitrate, p-hydroxybenzoic acid esters, polyhexamethylene biguanide (“PHMB”), potassium sorbate, propyl paraben, propylene glycol, sodium benzoate, sodium perborate, sodium propionate, sorbic acid, stabilized thimerosal, and/or thimerosal.

The pharmaceutical compositions of the present invention may also contain pH adjusting agents or neutralizing agents. The pH adjusting agent or neutralizing agents is selected from the group consisting of sodium hydroxide, potassium hydroxide, magnesium hydroxide, sodium carbonate, Tris, sodium linoleate, sodium oleate, potassium carbonate, potassium linoleate, potassium oleate, hydrochloric acid and mixtures thereof.

In certain non-limiting embodiments, the ready-to-use dexmedetomidine composition is formulated at a pH of between about 4 and about 8, or between about 4 and about 7. In other non-limiting embodiments, the ready-to-use dexmedetomidine composition is formulated at a pH of between about 4.7 and about 6.2. In a preferred non-limiting embodiment, the ready-to-use dexmedetomidine composition is formulated at a pH of between about 4.5 to about 8.0, more preferably at a pH of between about 4.5 to about 7.0.

In a further embodiment, the present application provides storage stable, ready-to-use composition comprising dexmedetomidine or a pharmaceutically acceptable salt thereof and an antioxidant in a polymeric container.

In a further embodiment, the present application provides storage stable, ready-to-use composition comprising dexmedetomidine or a pharmaceutically acceptable salt thereof, a tonicity adjusting agent and an antioxidant in a polymeric container.

In a further embodiment the present application provides storage stable, ready-to-use composition comprising dexmedetomidine or a pharmaceutically acceptable salt thereof, a tonicity adjusting agent and methionine, in a polymeric container comprising a cyclic olefin.

In other non-limiting embodiments, the ready-to-use dexmedetomidine composition comprises a tonicity adjusting agent that is sodium chloride or dextrose.

In other non-limiting embodiments, the ready-to-use dexmedetomidine composition comprises dexmedetomidine mixed or dissolved in a sodium chloride saline solution. The saline solution can comprise sodium chloride present at a concentration of between about 0.05 weight percent and about 2 weight percent, or between about 0.05 weight percent and about 1 weight percent. In one preferred, non-limiting embodiment, the sodium chloride is present at a concentration of about 0.9 weight percent.

In other non-limiting embodiments, the ready-to-use dexmedetomidine composition of the present invention comprises dexmedetomidine, or a pharmaceutically acceptable salt thereof, at a concentration of about 4 μg/mL and sodium chloride at a concentration of about 0.90 weight percent.

In yet another embodiment, the present application provides storage stable, ready-to-use composition comprising dexmedetomidine or a pharmaceutically acceptable salt thereof, a tonicity adjusting agent and methionine in a polymeric container, where the polymer that is in contact with the composition comprises cyclic olefin copolymer.

As used herein, the term “cyclic olefin polymer” or “COP” includes polymers that are made from at least one cyclic monomer. The term “cyclic olefin polymer” also includes cyclic olefin copolymers (“COC”). Suitable cyclic olefin polymers and copolymers may be used, including commercially-available cyclic olefin copolymers based on a variety of different types of cyclic monomers and polymerization methods. In some embodiments, the cyclic olefin polymer is a clear resin. In certain embodiments, the polymeric container or vessel includes cyclic olefin copolymer container, more specifically CZ® (Crystal Zenith) resin containers and syringes (available form West Pharmaceutical Services, Inc.). In certain non-limiting embodiments, polymeric container comprises of laminated bags having an inner layer comprising a cyclic olefin copolymer (POLYELITE® PHC-three layered bags-commercially available from Hosokawa Micron Corporation, Japan).

Several types of commercial cyclic olefin copolymers based on different types of cyclic monomers and polymerization methods may be used. In certain embodiments, the cyclic olefin copolymers are produced by chain copolymerization of cyclic monomers such as 8,9,10-trinorborn-2-ene (norbornene) or 1,2,3,4,4a,5,8,8a-octahydro-1,4:5,8-dimethanonaphthalene (tetracyclododecene) with ethene, such as Ticona's TOPAS, Mitsui Chemical's APEL. In certain embodiments, the cyclic olefin copolymers are produced by ring-opening metathesis polymerization of various cyclic monomers followed by hydrogenation, as in Japan Synthetic Rubber's ARTON, Zeon Chemical's Zeonex and Zeonor. See, e.g., J. Y. Shin, et al., “Chemical Structure And Physical Properties Of Cyclic Olefin Copolymers,” Pure and Applied Chemistry, 77:801-814 (2005).

By “sealed polymeric container,” “sealed container” or “finished product” is meant that the pharmaceutical composition is provided inside a sealed container. At least the part of the container that is in contact with the pharmaceutical composition or active ingredient comprises a polymeric cyclic olefin material. In certain embodiments, the entire container is a polymeric cyclic olefin material.

In certain embodiments, the container is a vial, pre-filled syringe, bag (e.g., an infusion bag or a pouch), bottle, or ampoule. In certain non-limiting embodiments, the polymeric container or vessel includes flexible polymeric containers such as bags, e.g., infusion bags or pouches.

Suitable flexible polymeric containers may comprise from 3 to 7 layers, for example. These flexible polymeric containers may be made of copolymerized polyolefin and styrene with 3 layers (available from Terumo corporation, Japan). In certain non-limiting embodiments, these flexible polymeric containers may be made of polyethylene film composed of three coextruded layers (commercially available from Hosokawa Micron Corporation, Japan). In certain non-limiting embodiments, these flexible polymeric containers may be made of polypropylene film composed of three coextruded layers (commercially available from Technoflex, France). In certain non-limiting embodiments, the flexible polymeric container comprises of laminated bags having an inner layer comprising a cyclic olefin copolymer (POLYELITE® PHC-three layered bags-commercially available from Hosokawa Micron Corporation, Japan).

It has been observed that not all the polymeric containers are compatible with the dexmedetomidine composition of the present application. Some of the polymeric containers produce leachables when stored for more than a month contaminating the dexmedetomidine composition with unidentified chemicals. The polymeric container of the present application is selected such that it does not adsorb or degrade dexmedetomidine, nor does it produce leachables. The examples provided herein provide guidance in the selection of suitable polymeric containers. It may also be able to undergo heat sterilization without contaminating the dexmedetomidine solution.

Conventional methods may be used for making the ready-to-use pharmaceutical compositions described herein. For example, such methods may comprise: (i) combining one or more antioxidants or stabilizers with water for injection to form a first solution; (ii) adding dexmedetomidine or a pharmaceutically acceptable salt thereof to the first solution to provide a second solution; (iii) optionally, adding a tonicity adjusting agent to the second solution to provide a third solution; (iv) optionally, adjusting the pH of the third solution by adding hydrochloric acid or sodium hydroxide to provide a fourth solution; (v) filling one or more containers with the fourth solution; and (vi) sealing the containers; and optionally, sterilizing the containers. A pharmaceutically inert gas (e.g., which may be selected from nitrogen or carbon dioxide) may be bubbled into the solution to drive out oxygen.

In certain embodiments, these methods for making the compositions of the invention will comprise filling the solution into vials; and sealing the vials. For example, rubber stoppers or other suitable closures may be used for sealing the vials.

The formulations of the present invention may be sterilized using methods known to the skilled artisan. Non-limiting examples of sterilization techniques include filtration through a bacterial-retaining filter, terminal sterilization, incorporation of sterilizing agents, irradiation, and heating. In certain non-limiting embodiments, the dexmedetomidine composition of the present application are heat sterilized in the polymeric container.

Sterilization may be accomplished by any of the conventional methods including aseptic filling, irradiation and heat sterilization. Heat sterilization is normally performed using steam, preferably wet steam to allow for the use of pressure as a means of temperature control. The time period for the sterilization must be long enough to meet the sterility requirements required of an injectable product. When steam is used, the period may be from about 5 to 30 minutes at temperatures of about 110° C. to 130° C., or from about 10 to 30 minutes at temperatures of about 110° C. to 130° C., preferably at 120° C. to 125° C. for 15 to 30 minutes. In another embodiment, the sterilization can be at 122° C. for 5 to 15 minutes.

In certain non-limiting embodiments, the ready-to-use dexmedetomidine composition of the present invention can be stored as a liquid in an aliquot having a total volume of about 20 mL. In certain non-limiting embodiments, the ready-to-use dexmedetomidine composition of the present invention can be stored as a liquid in an aliquot having a total volume of about 50 mL. In certain non-limiting embodiments, the ready-to-use dexmedetomidine composition of the present invention can be stored as a liquid in an aliquot having a total volume of about 100 mL.

The present invention provides for compositions in single-dose and/or multi-dose formulations. In some embodiments, the containers may be in the range of about 0.1 to 500 mL in volume, preferably in the range of about 0.5 to 250 mL, more preferably in the range of about 1 to 100 mL, and most preferably in the range of about 10 to 50 mL. In some embodiments, the composition may exist in a 2 mL, 5 mL, 10 ml, 20 mL, 50 mL, 100 mL or 200 mL container.

In certain embodiments, the finished product is provided as a container having an amount of dexmedetomidine (eq.) of 80 μg/20 mL, 200 μg/50 mL or 400 μg/100 mL.

In some embodiments, the container may be a single-dose formulation, or a multi-dose formulation. For example, a pre-filled syringe according to the invention may include single use auto injectors, or reusable auto injectors. In some embodiments, the same container may be used for multiple applications of the composition for up to about 10 days after initial use, preferably up to about 15 days, more preferably up to about 30 days, more preferably up to about 45 days, and most preferably up to about 60 days.

In certain non-limiting embodiments, ready-to-use dexmedetomidine compositions of the present application are useful in perioperative care of a patient or for sedation.

### EXAMPLES

The following examples are exemplary and not intended to be limiting. The above disclosure provides many different embodiments for implementing the features of the invention, and the following examples describe certain embodiments. Other modifications and methods known to one of ordinary skill in the art can also be applied to the following experimental procedures, without departing from the scope of the invention.

### General HPLC Procedure

As explained in detail below, the following HPLC procedure can be used to detect and quantify impurities of dexmedetomidine as well as assay calculation. The materials and general conditions are listed below:

TABLE 1
Chromatographic conditions

| | |
|---|---|
| Chromatographic | Gradient |
| Column | Luna Omega 5 μm PS C18 100 A°, |
| | 250 × 4.6 mm or equivalent |
| Wavelength | 220 nm |
| Flow rate | 1.0 mL/min |
| Injection volume | 100 μL |
| Column temperature | 25° C. |
| Retention time | approximately 9.2 minutes for Dexmedetomidine |
| | peak |
| Run time | 40 minutes |
| Needle Wash | Mixture of Water:Methanol in the ratio of 20:80 v/v |
| Mobile Phase A | To 1000 mL of water add 1 mL of Orthophosphoric |
| | acid and mix well. |
| Mobile Phase B | Mix Water and Acetonitrile in the ratio of 300:700 |
| | v/v. To this add 1 mL of Orthophosphoric acid |
| | and mix well. |

TABLE 2
Gradient Program

| Time (min) | % Mobile Phase A | % Mobile Phase B |
|---|---|---|
| 0.01 | 75 | 25 |
| 30 | 35 | 65 |
| 32 | 75 | 25 |
| 40 | 75 | 25 |

The relative retention times (RRTs) of the related substances with respect to dexmedetomidine peaks are shown in the Table 3 below.

TABLE 3
RRT for Dexmedetomidine Impurities

| S. | | | |
|---|---|---|---|
| No. | Name of Impurity | Structure | RRT* |
| 1 | Dexmedetomidine Impurity 1 1-(4-imidazoyl)-1-(2,3- dimethylphenyl) ethylene | 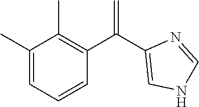 | 1.11 |
| 2 | N-Benzyl-hydroxymedetomidine 1-(1-Benzyl-1H-imidazol-5-yl)-1- (2,3-dimethylphenyl) ethanol | 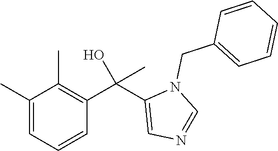 [SMILES: Cc1cccc(C(C)(O)c2cncn2Cc2ccccc2)c1C] | 1.56 |
| 3 | N-Benzyl-medetomidine 1-benzyl-5-(1-(2,3-dimethyl- phenyl)ethyl)-1H-imidazole | 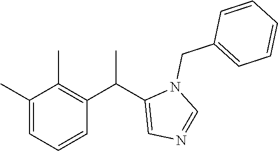 [SMILES: Cc1cccc(C(C)c2cncn2Cc2ccccc2)c1C] | 2.07 |
| 4 | N-Benzyl-vinyl analogue 1-benzyl-5-[1-(2,3-dimethyl- phenyl)vinyl]-1H-imidazole | 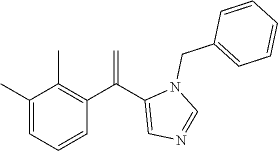 [SMILES: C=C(c1cccc(C)c1C)c1cncn1Cc1ccccc1] | 2.18 |
| 5 | Ethyl-medetomidine 5-[1-(2,3-dimethylphenyl) ethyl]-1H-imidazole | 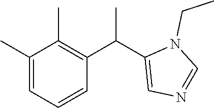 [SMILES: CCn1cncc1C(C)c1cccc(C)c1C] | 1.24 |
| 6 | Hydroxy-medetomidine 1-(2.3-Dimethylphenyl)-1-(1H- imidazol-5-yl)ethanol | 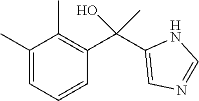 [SMILES: Cc1cccc(C(C)(O)c2cnc[nH]2)c1C] | 0.56 |

### Example 1

Dexmedetomidine Hydrochloride Injection (Eq. 4 mcg/mL), Using L-methionine

| | | |
|---|---|---|
| Ingredients | Qty/mL | Qty/Batch |

| | | | | |
|---|---|---|---|---|
| Dexmedetomidine hydrochloride | 0.00472 | mg** | 0.00472 | g |
| Sodium chloride | 9.0 | mg | 9.0 | g |
| L-Methionine | 1.0 | mg | 1.0 | g |
| Water for Injection | Qs to 1 | mL | 1000 | mL |

**Each ml contains Dexmedetomidine hydrochloride equivalent to 4 mcg (0.004 mg) of dexmedetomidine

About 80% of the water for injection was added into the manufacturing vessel under nitrogen purging. Sodium chloride, followed by L-methionine, was added in portions and mixed until a clear solution was formed. Dexmedetomidine HCl was added to the clear solution, and mixing continued until there was complete dissolution. A sufficient quantity of water was added to make up the final volume.

The final solution was passed through a 0.22μ membrane filter, and then filled into 20 mL CZ vials with the target volume. A portion of these vials was sterilized at 122.1±1° C. for 15 min. The sterilized vials were observed for stability at various temperature conditions and results are provided in Table 4.

TABLE 4
Stability observations

| | | 60° C. | | 40° C. ± 2° C./ | | | |
|---|---|---|---|---|---|---|---|
| | | (Stress) | | 75% RH ± 5% RH | | | |
| Parameters | Initial | 15 D | 1 M | 1 M | 2 M | 3 M | 6 M |
| pH | 5.55 | NA | 5.65 | 5.63 | 5.61 | 5.32 | 5.56 |
| % Assay of | 94.47 | 96.9 | 93.4 | 93.7 | 93.6 | 93.1 | 93.3 |
| Dexmedetomidine | | | | | | | |
| Total Related | ND | ND | ND | ND | ND | ND | ND |
| Substances (%) | | | | | | | |

ND: Not detected,
D: Days,
M: Month,
NA: Not Analysed

### Example 2

Dexmedetomidine Hydrochloride Injection (eq. 4 mcg/mL), Using EDTA Sodium

| | | |
|---|---|---|
| Ingredients | Qty/mL | Qty/Batch |

| | | | | |
|---|---|---|---|---|
| Dexmedetomidine hydrochloride | 0.00472 | mg** | 0.00472 | g |
| Sodium Chloride | 9 | mg | 9.0 | g |
| EDTA Sodium | 0.06 | mg | 0.06 | g |
| WFI | Qs to 1 | ml | 1000 | mL |

**Each ml contains Dexmedetomidine hydrochloride equivalent to 4 mcg (0.004 mg) of dexmedetomidine

About 80% of the water for injection was added into the manufacturing vessel under nitrogen purging. Sodium chloride, followed by EDTA sodium, was added in portions and mixed until a clear solution was formed. Dexmedetomidine HCl was added to the clear solution and mixing continued until there was complete dissolution. A sufficient quantity of water was added to make up the final volume.

The final solution was passed through a 0.22μ membrane filter and then filled into 20 mL, 50 mL and 100 mL CZ vials, respectively, with the target volume. A portion of these vials was sterilized at 122.1±1° C. for 15 min. The sterilized vials were observed for stability at various temperature conditions and results are provided in Table 5.

TABLE 5
Stability observations

| | | 60° C. (Stress) | | 40° C. ± 2° C./75% RH ± 5% RH | | | |
|---|---|---|---|---|---|---|---|
| Parameters | Initial | 15 D | 1 M | 1 M | 2 M | 3 M | 6 M |
| pH | 5.94 | NA | 6.06 | 6.04 | 6.04 | 5.96 | 6.02 |
| % Assay of | 104.2 | 106.6 | 102.8 | 102.8 | 103.3 | 102.8 | 102.7 |
| Dexmedetomidine | | | | | | | |
| Total Related | ND | 0.41 | 0.46 | 0.26 | 0.39 | 0.46 | 0.57 |
| Substances (%) | | | | | | | |

ND: Not detected,
D: Days,
M: Month,
NA: Not Analysed

### Example 3

Dexmedetomidine Hydrochloride Injection (eq. 4 mcg/mL), Using Propylene Glycol

| | | |
|---|---|---|
| Ingredients | Qty/mL | Qty/Batch |

| | | | | |
|---|---|---|---|---|
| Dexmedetomidine hydrochloride | 0.00472 | mg** | 0.00472 | g |
| Sodium Chloride | 9 | mg | 9.0 | g |
| Propylene glycol | 1.02 | mg | 1.02 | g |
| WFI | Qs to 1 | ml | 1000 | mL |

**Each ml contains Dexmedetomidine hydrochloride equivalent to 4 mcg (0.004 mg) of dexmedetomidine

About 80% of the water for injection was added into the manufacturing vessel under nitrogen purging. Sodium chloride, followed by propylene glycol, was added in portions and mixed until a clear solution was formed. Dexmedetomidine HCl was added to the clear solution and mixing continued until there was complete dissolution. A sufficient quantity of water was added to make up the final volume.

The final solution was passed through a 0.22μ membrane filter and then filled into 20 mL, 50 mL and 100 mL CZ vials, respectively, with the target volume. A portion of these vials was sterilized at 122.1±1° C. for 15 min. The sterilized vials were observed for stability at various temperature conditions and results are provided in Table 6.

TABLE 6
Stability observations

| | | 60° C. (Stress) | | 40° C. ± 2° C./75% RH ± 5% RH | | | |
|---|---|---|---|---|---|---|---|
| Parameters | Initial | 15 D | 1 M | 1 M | 2 M | 3 M | 6 M |
| pH | 5.49 | NA | 5.54 | 5.58 | 5.51 | 5.54 | 5.37 |
| % Assay of | 104.2 | 106.4 | 102.6 | 102.5 | 102.9 | 102.4 | 103.4 |
| Dexmedetomidine | | | | | | | |
| Total Related | 0.26 | 0.33 | 0.24 | 0.24 | 0.36 | 0.36 | 3.67 |
| Substances (%) | | | | | | | |

ND: Not detected,
D: Days,
M: Month,
NA: Not Analysed

### Example 4

Dexmedetomidine Hydrochloride Injection (eq. 4 mcg/mL), Using Glycerol

| | | |
|---|---|---|
| Ingredients | Qty/mL | Qty/Batch |

| | | | | |
|---|---|---|---|---|
| Dexmedetomidine hydrochloride | 0.00472 | mg** | 0.00472 | g |
| Sodium Chloride | 9 | mg | 9.0 | g |
| Glycerol | 1.33 | mg | 1.33 | g |
| WFI | Qs to 1 | ml | 1000 | mL |

**Each ml contains Dexmedetomidine hydrochloride equivalent to 4 mcg (0.004 mg) of dexmedetomidine

About 80% of the water for injection was added into the manufacturing vessel under nitrogen purging. Sodium chloride, followed by glycerol, was added in portions and mixed until a clear solution was formed. Dexmedetomidine HCl was added to the clear solution and mixing continued until there was complete dissolution. A sufficient quantity of water was added to make up the final volume.

The final solution was passed through a 0.22μ membrane filter and then filled into 20 mL, 50 mL and 100 mL CZ vials, respectively, with the target volume. A portion of these vials was sterilized at 122.1±1° C. for 15 min. The sterilized vials were observed for stability at various temperature conditions and results are provided in Table 7.

TABLE 7
Stability observations

| | | 60° C. | | 40° C. ± 2° C./ | | | |
|---|---|---|---|---|---|---|---|
| | | (Stress) | | 75% RH ± 5% RH | | | |
| Parameters | Initial | 15 D | 1 M | 1 M | 2 M | 3 M | 6 M |
| pH | 5.49 | NA | 5.54 | 5.55 | 5.51 | 5.52 | 5.45 |
| % Assay of Dexmedetomidine | 103.3 | 105.1 | 102 | 102 | 103 | 102.5 | 102.3 |
| Total Related Substances (%) | ND | ND | ND | ND | 0.09 | 0.17 | 0.11 |

ND: Not detected,
D: Days,
M: Month

### Example 5

Dexmedetomidine Hydrochloride Injection (eq. 4 mcg/mL), Using L-Methionine

| | | |
|---|---|---|
| Ingredients | Qty/mL | Qty/Batch |

| | | | | |
|---|---|---|---|---|
| Dexmedetomidine hydrochloride | 0.00472 | mg** | 0.710 | g |
| Sodium chloride | 9.0 | mg | 1350.0 | g |
| L-Methionine | 1.0 | mg | 150.39 | g |
| Water for Injection | Qs to 1 | mL | 150 | L |

**Each ml contains Dexmedetomidine hydrochloride equivalent to 4 mcg (0.004 mg) of dexmedetomidine

About 90% of the water for injection was added into the manufacturing vessel under nitrogen purging. Sodium chloride, followed by L-methionine, was added in portions and mixed until a clear solution was formed. Dexmedetomidine HCl was added to the clear solution and mixing continued until there was complete dissolution. A sufficient quantity of water was added to make up the final volume.

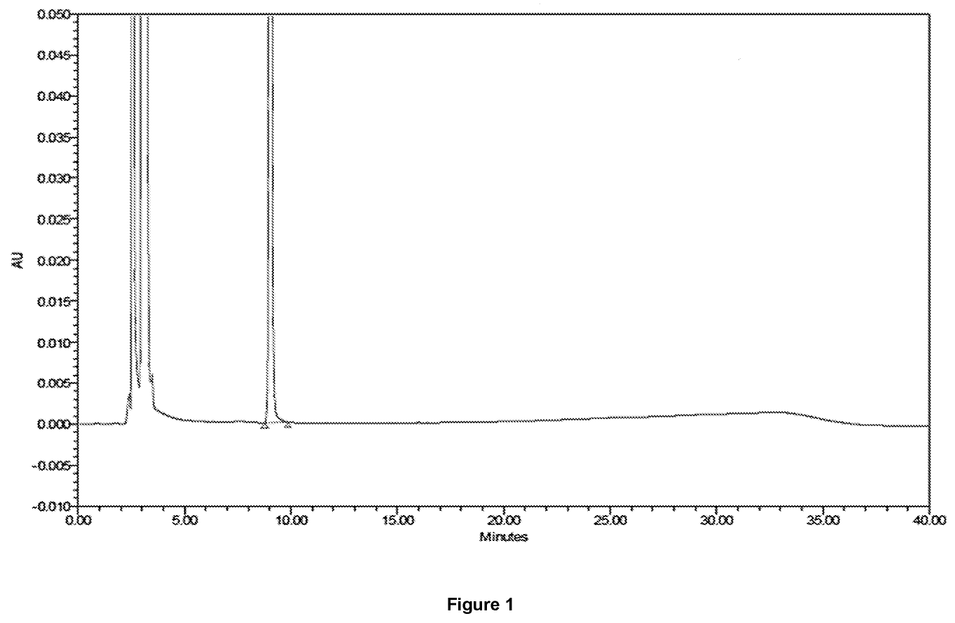

The final solution was passed through a 0.22μ membrane filter and then filled into 100 mL CZ vials, with the target volume. A portion of these vials was sterilized at 122.1±1° C. for 15 min. The sterilized vials were observed for stability at various temperature conditions and results are provided in Table 8, as well as in FIG. 1.

TABLE 8
Stability observations

| | | | | | 25° C. ± 2° C./ | |
|---|---|---|---|---|---|---|
| | | 40° C. ± 2° C./ | | | 60% RH ± | |
| | | 75% RH ± 5% RH | | | 5% RH | |
| Parameters | Initial | 1 M | 3 M | 6 M | 3 M | 6 M |
| pH | 5.8 | 5.7 | 5.7 | 5.7 | 5.7 | 5.7 |
| % Assay of | 100.0% | 99.9% | 98.6% | 100.4% | 99.1% | 100.7% |
| Dexmedeto- | | | | | | |
| midine | | | | | | |
| Total Related | ND | ND | ND | ND | ND | ND |
| Substances (%) | | | | | | |
| Total | <0.1% | <0.1% | <0.1% | <0.1% | <0.1% | <0.1% |
| Leachable | | | | | | |
| Impurities (%) | | | | | | |

ND: Not detected,
D: Days,
M: Month

### Example 6

Dexmedetomidine Hydrochloride Injection (eq. 4 mcg/mL), Using L-Methionine Filled in Polymeric Containers

| Ingredients | Qty/mL | | Qty/Batch | |
|---|---|---|---|---|
| Dexmedetomidine hydrochloride | 0.00472 | mg** | 23.62 | mg |
| Sodium chloride | 9.0 | mg | 45.0 | g |
| L-Methionine | 1.0 | mg | 5.0 | g |
| Water for Injection | q.s to 1 | mL | q.s to 2000 | mL |

**Each ml contains Dexmedetomidine hydrochloride equivalent to 4 mcg (0.004 mg) of dexmedetomidine

About 1.8 L of the water for injection was added into the manufacturing vessel under nitrogen purging at 20-25° C. Sodium chloride, followed by L-methionine, was added in portions and mixed until a clear solution was formed. Dexmedetomidine HCl was added to the clear solution and mixing continued until complete dissolution. A sufficient quantity of water was added to make up the final volume. The observed pH of the bulk solution was about 6.20

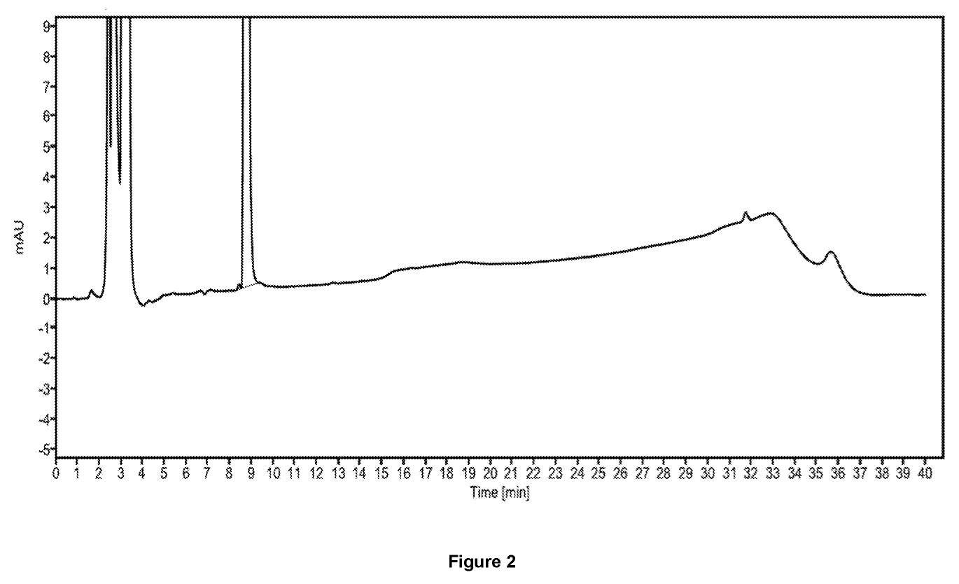

The final solution was passed through 0.22μ membrane filter and the filtered solution was filled into POLYELITE® PHC polymeric containers with 100 mL fill volume. (The POLYELITE® PHC containers comprise three layers, including an outside layer of polypropylene, a middle layer of linear low density polyethylene, and an inner layer (i.e., the contact layer) of cyclic olefin polymer.) These containers were sterilized at 122±1° C. for 15 min. The sterilized containers were observed for stability at various temperature conditions and results are provided in Table 9, as well as in FIG. 2.

TABLE 9
POLYELITE ® PHC Bag (Hosokawa)

| | | 1 Month | 1 Month | 3 Month |
|---|---|---|---|---|
| | | 25 ± 2° C./ | 40 ± 2° C./ | 40 ± 2° C./ |
| Parameters | Initial | 60% RH | 75% RH | 75% RH |
| pH | 5.96 | 6.23 | 5.78 | 6.02 |
| % Assay of | 99.8 | 103.0 | 103.0 | 99.5 |
| Dexmedetomidine | | | | |
| % Assay of | 103.9 | 102.6 | 102.9 | 101.4 |
| Methionine | | | | |
| Total Related | ND | ND | ND | ND |
| Substances (%) | | | | |
| Total Leachable | <0.1% | <0.1% | <0.1% | <0.1% |
| Impurities (%) | | | | |

ND: Not detected

### Comparative Examples

Dexmedetomidine Hydrochloride Injection (eq. 4 mcg/mL), Using L-Methionine Filled in Polymeric Containers

| Ingredients | Qty/mL | | Qty/Batch | |
|---|---|---|---|---|
| Dexmedetomidine hydrochloride | 0.00472 | mg** | 23.62 | mg |
| Sodium chloride | 9.0 | mg | 45.0 | g |
| L-Methionine | 1.0 | mg | 5.0 | g |
| Water for Injection | q.s to 1 | mL | q.s to 5000 | mL |

**Each ml contains Dexmedetomidine hydrochloride equivalent to 4 mcg (0.004 mg) of dexmedetomidine

About 4.8 L of the water for injection was added into the manufacturing vessel under nitrogen purging at 20-25° C. Sodium chloride, followed by L-methionine, was added in portions and mixed until a clear solution was formed. Dexmedetomidine HCl was added to the clear solution and mixing continued until there was complete dissolution. A sufficient quantity of water was added to make up the final volume. The observed pH of the bulk solution was about 5.94.

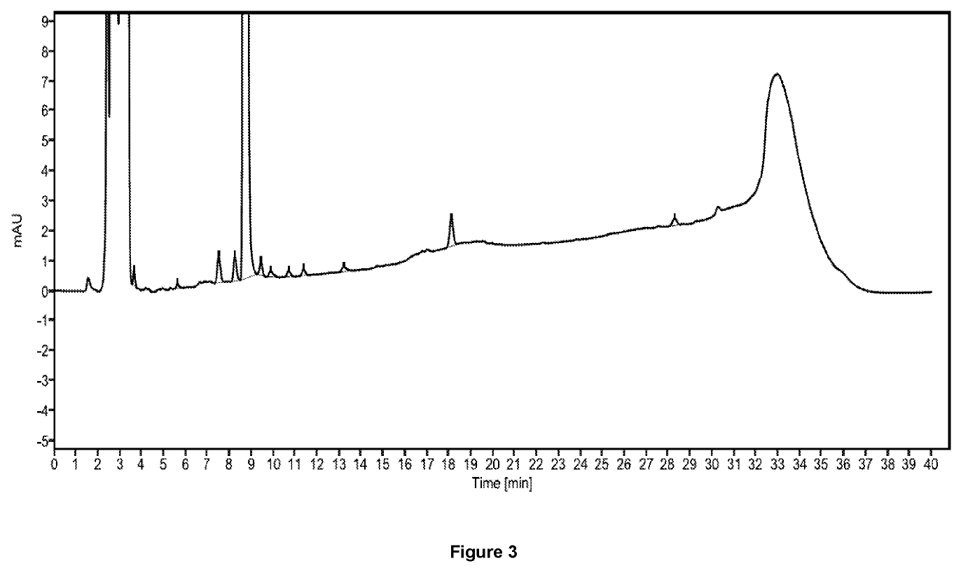

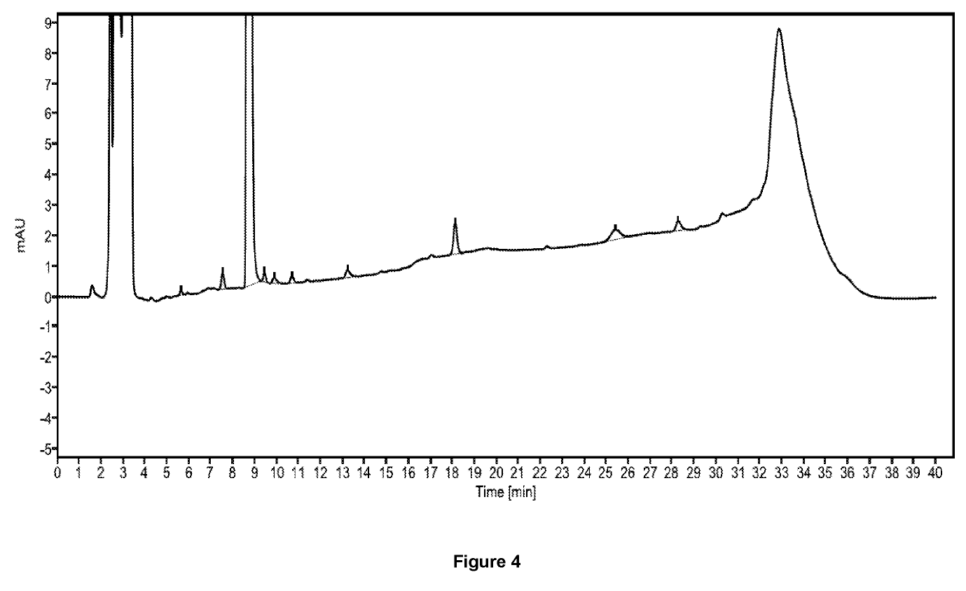

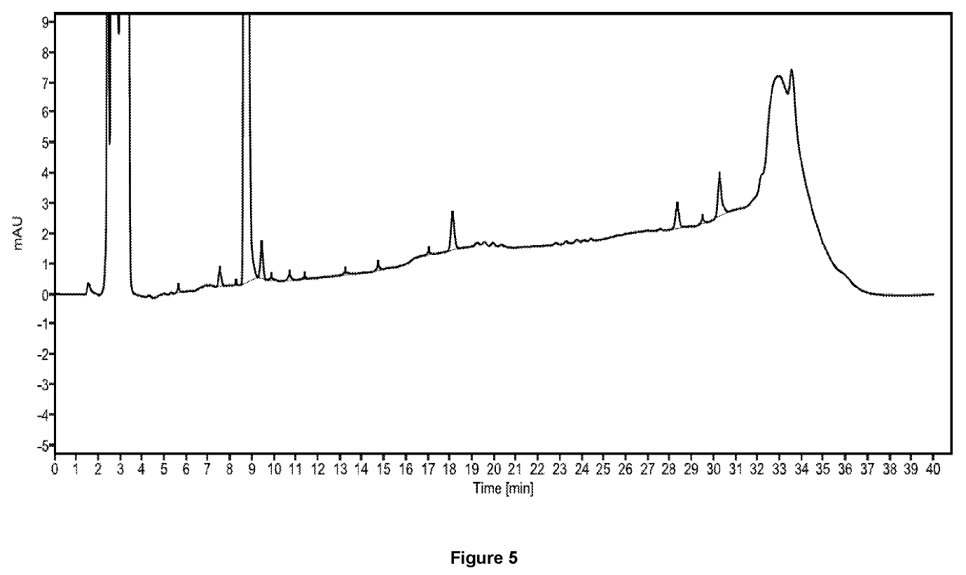

The final solution was passed through 0.22μ membrane filter and then split into portions. Each portion of the filtered solution was filled into different kinds of polymeric containers with 52 ml fill volume, as specified in Tables 10-12. A portion of these containers were sterilized at 122.1±1° C. for 15 min. The sterilized containers were observed for stability at various temperature conditions, and results are summarized in Tables 10-12, as well as in FIGS. 3-5.

TABLE 10
Three-layer Polyolefin/Styrene-block copolymer Bag (Terumo)

| | | 1 Month | 3 Month | 1 Month | 3 Month |
|---|---|---|---|---|---|
| | | 25 ± 2° C./ | 25 ± 2° C./ | 40 ± 2° C./ | 40 ± 2° C./ |
| Parameters | Initial | 60% RH | 60% RH | 75% RH | 75% RH |
| pH | 6.09 | 5.80 | 6.32 | 5.91 | 6.21 |
| % Assay of | 97.7 | 98.7 | 102.1 | 98.9 | 101.4 |
| Dexmedetomidine | | | | | |
| % Assay of | 101.6 | 102.5 | 103.4 | 102.7 | 103.4 |
| Methionine | | | | | |
| Total Related | ND | ND | ND | ND | 0.75 |
| Substances (%) | | | | | |
| Total Leachable | 1.04 | 1.07 | 1.10 | 1.64 | 3.11 |
| Impurities (%) | | | | | |

TABLE 11
Polypropylene Bag (Technoflex)

| | | 1 Month | 3 Month | 1 Month | 3 Month |
|---|---|---|---|---|---|
| | | 25 ± 2° C./ | 25 ± 2° C./ | 40 ± 2° C./ | 40 ± 2° C./ |
| Parameters | Initial | 60% RH | 60% RH | 75% RH | 75% RH |
| pH | 5.87 | 5.85 | 6.08 | 5.84 | 6.05 |
| % Assay of | 98.6 | 98.8 | 102.2 | 99.2 | 101.9 |
| Dexmedetomidine | | | | | |
| % Assay of | 103.2 | 102.6 | 103.6 | 102.7 | 103.4 |
| Methionine | | | | | |
| Total Related | ND | ND | ND | ND | ND |
| Substances (%) | | | | | |
| Total Leachable | 0.54 | 1.64 | 1.44 | 1.47 | 3.12 |
| Impurities (%) | | | | | |

ND: Not Detected

TABLE 12
POLYELITE TH82 Film (Polyethylene bag-Hosokawa)

| | | 1 Month | 3 Month | 1 Month | 3 Month |
|---|---|---|---|---|---|
| | | 25 ± 2° C./ | 25 ± 2° C./ | 40 ± 2° C./ | 40 ± 2° C./ |
| Parameters | Initial | 60% RH | 60% RH | 75% RH | 75% RH |
| pH | 6.68 | 6.10 | 6.16 | 6.08 | 6.15 |
| % Assay of | 97.7 | 98.6 | 102.3 | 98.6 | 101.0 |
| Dexmedetomidine | | | | | |
| % Assay of | 102.0 | 100.9 | 103.5 | 102.7 | 103.4 |
| Methionine | | | | | |
| Total Related | ND | ND | ND | ND | ND |
| Substances (%) | | | | | |
| Total Leachable | 0.49 | 1.58 | 1.52 | 2.33 | 3.82 |
| Impurities (%) | | | | | |

ND: Not Detected

Having now fully described this invention, it will be understood by those of ordinary skill in the art that it can be performed within a wide equivalent range of parameters without affecting the scope of the invention or any embodiment thereof. All publications, patent applications and patents disclosed herein are incorporated by reference in their entirety.

1. A ready-to-use, injectable pharmaceutical composition comprising (i) dexmedetomidine or a pharmaceutically acceptable salt thereof, (ii) at least one pharmaceutically acceptable excipient and (iii) at least one pharmaceutically acceptable liquid vehicle, wherein the composition is provided in a sealed container, and wherein the sealed container comprises a cyclic olefin polymer.

2. The pharmaceutical composition according to claim 1, wherein the sealed container comprises a cyclic olefin polymer in at least one contact layer, wherein the contact layer is in direct contact with the pharmaceutical composition.

3. The pharmaceutical composition according to claim 1, wherein the sealed container selected from the group consisting of vials, pre-filled syringes, bags, bottles and ampoules.

4. The pharmaceutical composition according to claim 1, wherein cyclic olefin is a polymer selected from the group consisting of cyclic olefin copolymers, cyclic olefin polymers, and mixtures thereof.

5. The pharmaceutical composition according to claim 1, comprising dexmedetomidine hydrochloride.

6. The pharmaceutical composition according to claim 1, wherein the dexmedetomidine is present at a concentration of about 4 μg/mL.

7. The pharmaceutical composition according to claim 1, wherein the composition is formulated at a pH of between about 4.5 and about 7.0.

8. The pharmaceutical composition according to claim 1, further comprising an antioxidant.

9. The pharmaceutical composition according to claim 8, wherein the antioxidant is provided in a stabilizing amount.

10. The pharmaceutical composition according to claim 1, wherein the composition comprises a stabilizing amount of at least one pharmaceutically acceptable excipient selected from the group consisting of methionine, glycerol, propylene glycol, phenol, EDTA, BHT, and mixtures thereof.

11. The pharmaceutical composition of claim 1, comprising:
(i) about 4 μg/mL of dexmedetomidine hydrochloride;
(ii) about 9.0 mg/mL of sodium chloride;
(iii) about 1.0 mg/mL of L-methionine; and
(iv) water for injection.

12. The pharmaceutical composition according to claim 1, wherein the composition is stable for 6 months when stored at ambient conditions.

13. The pharmaceutical composition according to claim 1, wherein the composition is stable for at least 3 months at 25° C. and 60% relative humidity.

14. A ready-to-use, injectable pharmaceutical composition comprising (i) dexmedetomidine or a pharmaceutically acceptable salt thereof, (ii) at least one antioxidant, and (iii) at least one pharmaceutically acceptable liquid vehicle.

15. The pharmaceutical composition according to claim 14, wherein the composition is provided in a sealed container, and wherein the sealed container comprises a cyclic olefin polymer.

16. The pharmaceutical composition according to claim 14, wherein the antioxidant is provided in an amount of about 0.05 mg/mL to about 5 mg/mL.

17. The pharmaceutical composition according to claim 14, wherein the antioxidant is provided in a stabilizing amount.

18. The pharmaceutical composition of claim 14 comprising:
(i) about 0.005 μg/mL to about 10 μg/mL of dexmedetomidine or a pharmaceutically acceptable salt thereof; and
(ii) about 0.05 mg/mL to about 2 mg/mL of an antioxidant.

19. The pharmaceutical composition of claim 14, wherein the composition further comprises a tonicity adjusting agent.

20. The pharmaceutical composition of claim 19, comprising:
(i) about 4 μg/mL of dexmedetomidine hydrochloride;
(ii) about 9.0 mg/mL of sodium chloride;
(iii) about 1.0 mg/mL of L-methionine; and
(iv) water for injection.

21. A method of treatment, comprising administering the pharmaceutical composition according to claim 1 to a patient in need thereof, for perioperative care or for sedation.

22. A method for making a ready-to-use, injectable pharmaceutical composition according to claim 1, comprising:
(i) combining one or more antioxidants or stabilizers with water for injection to form a first solution;
(ii) adding dexmedetomidine or a pharmaceutically acceptable salt thereof to the first solution to provide a second solution;
(iii) optionally, adding a tonicity adjusting agent to the second solution to provide a third solution;
(iv) optionally, adjusting the pH of the third solution by adding hydrochloric acid or sodium hydroxide to provide a fourth solution;
(iv) filling one or more containers with the fourth solution;
(v) sealing the containers; and
(vi) optionally, sterilizing the containers.
